SEQUENCE LISTING (10 sequences)

SEQ ID NO: 1 - DNA, 24 bp, Mycobacteria-N
ggagggttgggatgaacaaagcag

SEQ ID NO: 2 - DNA, 25 bp, Mycobacteria-S
gtatccgtgtgtcttgacctatttg

SEQ ID NO: 3 - DNA, 20 bp, Mycobacteria-M
gcagccaagaaggtagcgaa

SEQ ID NO: 4 - DNA, 18 bp, Mycobacteria-F
ccaagaaggcgacaaagg

SEQ ID NO: 5 - DNA, 18 bp, Mycobacteria-R
gacagctttcttggcggg

SEQ ID NO: 6 - DNA, 645 bp, hupB-M. tuberculosis, Rv2986c, Accession No. P95109
atgaacaaagcagagctcattgacgtgctcacacagaaattgggctcggaccgtcggcaggcgaccgccgccgtcgagaatgtcgttgacacgattgtgcgtgcggtacacaaaggcgacagcgtcaccattaccgggttcggtgtgttcgaacagcgtcgccgcgcggctcgagtggcccgcaatccgcgtaccggcgagacagtaaaggtgaagccgacgtcggtgccggcgttccgcccgggcgcgcaattcaaagcggttgtgtctggcgcgcagcgtctcccggcagaaggacccgctgttaagcgtggtgtgggggccagtgcagccaagaaggtagcgaagaaggcacctgccaagaaggcgacaaaggccgccaagaaggcggcgaccaaggcgcccgccaggaaggcggcgaccaaggcgcccgccaagaaagcggcgaccaaggcgcccgccaagaaagctgtcaaggccacgaagtcacccgccaagaaggtgaccaaggcggtgaagaagaccgcggtcaaggcatcggtgcgtaaggcggcgaccaaggcgccggcaaagaaggcagcggccaagcggccggctaccaaggctcccgccaagaaggcaaccgctcggcggggtcgcaaatag

SEQ ID NO: 7 - DNA, 618 bp, Hlp of Mycobacterium bovis, Accession No. Y18421
atgaacaaagcagagctcattgacgtgctcacacagaaattgggctcggaccgtcggcaggcgaccgccgccgtcgagaatgtcgttgacacgattgtgcgtgcggtacacaaaggcgacagcgtcaccattaccgggttcggtgtgttcgaacagcgtcgccgcgcggctcgagtggcccgcaatccgcgtaccggcgagacagtaaaggtgaagccgacgtcggtgccggcgttccgcccgggcgcgcaattcaaagcggttgtgtctggcgcgcagcgtctcccggcagaaggacccgctgttaagcgtggtgtgggggccagtgcagccaagaaggtagcgaagaaggcacctgccaagaaggcgacaaaggccgccaagaaggcggcgaccaaggcgcccgccaagaaagcggcgaccaaggcgcccgccaagaaagctgtcaaggccacgaagtcacccgccaagaaggtgaccaaggcggtgaagaagaccgcggtcaaggcatcggtgcgtaaggcggcgaccaaggcgccggcaaagaaggcagcggccaagcggccggctaccaaggctcccgccaagaaggcaaccgctcggcggggtcgcaaatag

SEQ ID NO: 8 - protein, 16 aa, Mycobacteria
VKPTSVPAFRPGAEFK

SEQ ID NO: 9 - DNA, 22 bp, Mycobacteria
gtgagcgacgggatttgcctat

SEQ ID NO: 10 - DNA, 21 bp, Mycobacteria
accacccaaaaccggatcgat

(19) United States
(12) Patent Application Publication
(10) Pub. No.: US 2006/0147931 A1
(43) Pub. Date: Jul. 6, 2006

(54) Characterization of hupb gene encoding histone like protein of mycobacterium tuberculosis

(75) Inventors: Krishna Prasad Hanumanthappa, New Delhi (IN); Savita Prabhakar, New Delhi (IN); Anjaki Mishra, New Delhi (IN); Tyagi Jaya Sivaswami, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: Department of Biotechnology, New Delhi (IN); All India Institute of Medical Sciences, New Delhi (IN)

(21) Appl. No.: 10/540,086

(22) PCT Filed: Sep. 9, 2003
(86) PCT No.: PCT/IN03/00302

§ 371 Date: Dec. 28, 2005

(30) Foreign Application Priority Data
Dec. 18, 2002 (IN) 1274/DEL/02

(51) Int. Cl. C12Q 1/68; C07H 21/04

(52) U.S. Cl. 435/6; 536/24.1

(57) ABSTRACT

Oligonucleotide primers for specific amplification of the hupB gene of Mycobacterium species selected from the group consisting of Seq ID No. 1, Seq ID No. 2, Seq ID No. 3, Seq ID No. 4, Seq ID No. 5 and a method for differentiating Mycobacterium species based on target gene encoding for histone like proteins such as hupB comprising of (a) obtaining DNA from culture or from clinical samples, b) amplifying a part of the target gene encoding for histone like proteins such as hupB of Mycobacterium species using DNA as a template in a polymerise chain reaction with a pair of oligonucleotide primers, c) detecting said amplified fragment of the hupB gene to detect the presence of Mycobacterial species or not and differentiating Mycobacterium tuberculosis from Mycobacterium bovis based on the size of the amplified fragment.

### FIELD OF THE INVENTION

This invention relates to the characterization of hupB gene encoding histone like protein of Mycobacterium tuberculosis. A method for differentiating M. tuberculosis and M. bovis based on the hupB gene.

### BACKGROUND OF THE INVENTION

Numerous techniques are in vogue to differentiate between members of the MTB complex. Several researchers have demonstrated that the use of IS6110 as a target for PCR amplification gives the best sensitivity and specificity in the diagnosis of tuberculosis. However when tested with standard mycobacterial species and strains obtained from ATCC and mycobacterial cultures isolated from clinical specimens, the target was limited in its ability to distinguish between M. tuberculosis complex from other mycobacteria.

Spoligotyping based detection of non-repetitive spacer sequences located between small repetitive units in the DR locus of the MTB complex strains, other genetic markers and biochemical tests have been used to differentiate between M. tuberculosis, M. bovis, M. africanum, M. microti, and M. canetti, (Niemann et al., 2000). Besides spoligotyping, mtp40 gene sequence (Liebana et al., 1996), pncA gene point mutation at position 169 (Scropio & Zhang, 1996), polymorphism of the oxyR locus (Sreevatsan et al., 1996), have been reported as useful targets for identification of the members of the TB complex.

Ideally, the target for PCR based detection should be that it discriminates not only among mycobacterial species but also is able to distinguish between closely related members of the MTB complex.

Although PCR techniques have been widely evaluated in the diagnosis of tuberculosis, the reports have generally focused on the detection of M. tuberculosis. However their success has been limited to differentiating between the tuberculosis complex and non-tuberculous mycobacteria.

Here we report a PCR assay that enables the precise identification of closely related mycobacteria belonging to the MTB complex. hupB gene encoding histone-like protein of M. tuberculosis has been exploited as a target for detection and differentiation of M. tuberculosis and M. bovis. The hupB gene target not only permits differentiation of M. tuberculosis from M. bovis, but also from among other members of the MTB complex, non-tuberculous mycobacteria as well as nor-mycobacterial species tested. The assay would prove useful especially in developing countries with a high incidence of infected livestock (Grange, 2001). M. bovis has been known to spread to humans from infected cattle by the aerosol route or by consumption of infected/contaminated dairy products (Zoonotic tuberculosis) (Moda et al., 1996; Cosivi et al., 1998). Although bovine tuberculosis had been largely eradicated in developed countries, recently resurgence of bovine tuberculosis has been reported (www.defra.gov.uk/animalh) and continues to occur in developing countries. The epidemiological impact of bovine tuberculosis on human health has not been assessed and is a major lacuna in developing countries. However with reports of tuberculosis due to M. bovis in AIDS patients (Bouvet et al., 1993; O'Reilly et al., 1995) and with increasing incidence of tuberculosis globally, rapid and reliable diagnostic assays are required not only for detection but also, identification of the pathogenic mycobacteria in clinical samples. This is essential for prompt diagnosis, treatment land control of tuberculosis. Identification of human pathogenic mycobacteria becomes all the more relevant with the need to develop alternate new generation vaccines for human use.

Immunogenecity of HupB protein: Two methods were used to identify mycobacterial constituents associated with human response namely the T cell blot and immuno—subtraction assays (Prabhakar et al., 1998). The 30 kDa fraction of the mycobacterial lysate was found to induce the highest lymphoproliferative index among the tuberculin reactors. In immuno-subtractive assays a prominent reactive band was similarly seen at approximately 30 kDa. The 30 kDa protein was electro-eluted from the SDS-PAGE gel and purified to homogeneity.

Using the internal peptide sequence, Seq. ID No. 8 (VKPTSVPAFRPGAQFK) a 100% identity was obtained with 16 amino acids of the cosmid CY349. The corresponding gene was later annotated and designated as the hupB gene (Rv 2986c, Cole et al., 1998). The protein was found to be localized in the cytoplasm and on the cytoplasmic surface of the mycobacterial membrane, by immuno-gold electron microscopy. The hupB gene has been classified among the DNA binding (histone like) proteins of M. tuberculosis (Cole et al., 1998). Primers were designed to amplify the hupB gene. A 645 bp amplicon was obtained in case, of M. tuberculosis. The α³²P labeled PCR amplicon was used in Southern hybridization to establish the size, prevalence and organization of the hupB gene in members of the MTB complex (M. tuberculosis and M. bovis) and other mycobacterial species.

### DRAWBACKS IN THE EXISTING STATE OF ART

Although PCR techniques have been widely evaluated in the diagnosis of tuberculosis, the reports have generally focused on the detection of M. tuberculosis. However their success has been limited to differentiating between the tuberculosis complex and non-tuberculous mycobacteria. Single-step PCR procedures to differentiate M. bovis from M. tuberculosis using IS6110 alone or in association with mtp40 gene have yielded discrepant results. Further it has been shown that mpt40 is not present in all M. tuberculosis strains and hence may not be useful for differentiating M. tuberculosis and M. bovis strains. The sen X3-regX3 intergenic region (IR) has been proposed as a target sequence for differentiating members of the MTB complex from other mycobacteria. However there are limitations in the use of this target region as it cannot identify members of the MTB complex, though BCG could be discerned from related strains. In the present study the hupB gene target has been shown to be a target, which permits differentiation of M. tuberculosis from M. bovis and from among other members of the TB complex, non-tuberculous mycobacterial and non-mycobacterial species tested. The assay would prove useful especially in developing countries with a high incidence of infected livestock (Grange, 2001). The epidemiological impact of bovine tuberculosis on human health has not been assessed and is a major lacuna in developing countries. However with reports of tuberculosis due to M. bovis in AIDS patients (Bouvet et al. 1993; O'Reilly et al, 1995) and with increasing incidence of tuberculosis globally, rapid and reliable diagnostic assays are required not only for detection but also identification of the pathogenic mycobacteria in clinical samples. This is essential for prompt diagnosis, treatment and control of tuberculosis. Here we report a PCR, RFLP and NESTED PCR assay that enables the precise identification of closely related mycobacteria belonging to the MTB complex.

### OBJECTS OF THE INVENTION

An object of this invention was:

(1) To characterize hupB gene encoding histone like protein of M. tuberculosis.

(2) To characterize mycobacterial gene as new target for novel anti-mycobacterial chemotheraputic agents.

(3) To differentiate between M. tuberculosis and M. bovis based on RFLP of the PCR generated amplicon of the hup B gene.

(4) To differentiate between M. tuberculosis and M. bovis based on the sequence of the PCR amplicon of the hup B gene.

(5) To differentiate between M. tuberculosis and M. bovis based on the nested PCR of the hup B gene.

### SUMMARY OF THE INVENTION

PCR, PCR-RFLP: The present invention relates to a process for differentiating Mycobacterial species using primers specific to the target of hupB gene encoding histone like protein. A single primer pair enables amplification of the target sequence from both Mycobacterium tuberculosis and Mycobacterium bovis. The size of the amplified product can differentiate Mycobacterium tuberculosis from Mycobacterium bovis and from related and unrelated species. DNA probes that hybridize to the amplified region distinguish Mycobacterium tuberculosis from Mycobacterium bovis and from related and unrelated species. Further there is provided a process (RFLP) for differentiating of the hupB gene of M. tuberculosis and M. bovis by restriction digestion of PCR amplified fragments.

NESTED PCR ASSAY: The utility of taking advantage of the hupB gene as a target is important from the point of developing molecular biological techniques to distinguish between members of the Mycobacterium tuberculosis complex. Owing to the current technical hurdles in identification of Mycobacterium tuberculosis and M. bovis, factual data on the prevalence of human or animal disease due to M. bovis is absent/limited. Such technology would help in establishing and documenting the authentic scenario of the extent of disease caused by M. bovis in live stock. The proposed NESTED PCR assay would be a major contribution to present day laborious techniques used to distinguish between Mycobacterium tuberculosis and M. bovis. In India compulsory elimination of tuberculin reactive animals cannot be practiced universally for religious and socioeconomic reasons. This assay would be an asset to epidemiology programs and in the identification of infected animals. The infected animals can then be segregated thereby limiting the spread of disease. Further with the extensive availability of dairy products (meat & milk) the NESTED PCR assay described here would help in alleviating the current problem of identification of human mycobacterial pathogens. These pathogens are members of the MTB complex and are genetically similar.

### FIGURE LEGENDS

FIG. 1 Position of the hupB gene and Primers used to generate PCR fragments.

Panel A: The position of the primers in the hupB sequence, which were used in order to obtain the PCR fragments have been depicted. Primer pairs N (Seq ID No. 1) & S (Seq ID No. 2) specific for the hupB gene; internal primer M (Seq ID No. 3) & S (Seq ID No. 2) specific for the C terminal part of the hupB gene.

Panel B, C and D: The ethidium bromide stained amplification fragments of M. tuberculosis and M. bovis generated using primer pairs N (Seq ID No. 1) & S (Seq ID No. 2) (Panel B), M (Seq ID No. 3) & S (Seq ID No. 2) (Panel C) and F(Seq ID No. 4) & R (Seq ID No. 1) (Panel D) were electrophoresed on polyacrylamide gels. The 645 and 618 bp (Panel B); 318 and 291 bp (Panel C); 116 and 89 bp (Panel D); fragments have been indicated. Lanes 1 & 4, 645 bp, 6 & 10, 318 bp, and 13, 116 bp of the of hupB gene/C terminal part of the gene amplification fragment obtained in M. tuberculosis H37Rv; lanes 2 & 5, 618 bp of hupB gene, 7 & 9, 291 bp and 11,12,15-17, 89 bp of the hupB gene/C terminal part of the gene amplification fragment obtained in M bovis AN5; 3, 8 & 14, 100 bp molecular weight markers.

FIG. 2 Specificity analysis of hupB_{M2} based PCR assay

Amplification fragments were electrophoresed on agarose gels. Their ethidium bromide staining (Panel A & Panel B) and hybridization profiles have been shown in Panel A′ & Panel B′ respectively. The 645 bp probe was used (generated by PCR using N (Seq ID No.1) and S (Seq ID No.2) primers and M. tuberculosis, DNA.). The 645 bp fragment has been indicated. Panels A & A′; Lanes 1 M. tuberculosis H37 Rv; 2, M. tuberculosis H37Ra; 3, M. bovis BCG; 4, M. microti; 5, M. xenopi; 6, M. fortuitum; 7, M. phlei; 8, M. gordonae; 9, M. vaccae 10, M. kansasii; 11, 100 bp Marker; 12, M. intracellulare; 13, M. avium; 14, M. scrofulaceum; 15, M. smegmatis; 16, M. tuberculosis P8497; 17, M. tuberculosis C1084; 18, M. tuberculosis 779634 19, M. chelonei; 20, M. tuberculosis P8473; 21, M. gastri.

Panel B & B′ Lanes 1, M. tuberculosis 1207; 2, E. coli; 3, N. asteroides; 4, S. aureus; 5, P. aeruginosa; 6, S. faecalis; 7, S. aureus; 8, A. niger; 9, A. fumigatus; 10, C. albicans; 11, 100 bp marker; 12, M. tuberculosis Erdman; 13, K. pneumoniae; 14, M. leprae; 15, M. africanum; 16, Negative control. Hybridisation in panels B & B′ was carried out with 645 bp fragment (PstI & NcoI digest from the plasmid pHLPMT).

FIG. 3 Sensitivity of detection of M. tuberculosis DNA by hupB based PCR assay.

Amplification reactions were performed with serial dilutions of M. tuberculosis DNA (1 ng to 1 fg). The ethidium bromide and hybridisation patterns are seen in panels A and B respectively. The 645 bp fragment has been indicated. Lanes 1, 1 ng; 2, 500 pg; 3, 50 pg; 4, 5 pg; 5, 1 pg; 6, 500 fg; 7, 100 fg; 8, 50 fg; 9, 10 fg 10, 5 fg; 11, 2 fg; 12, 1 fg; 13, Negative control; 14, positive control (M. tuberculosis) M, λ DNA HindIII digest. The detection limit was 50 pg by ethidium bromide staining and 500 fg for hybridisation.

FIG. 4 RFLP analysis of the 646 and 318 bp PCR fragments.

Panel A depicts the schematic representation of the position of the primers in the hupB sequence, which were used in order to obtain the 645 bp and 318 bp PCR fragments. Ethidium bromide staining for 645 bp (Panel B) and 318 bp (Panel C) amplification fragments are shown. Lanes 1, M. tuberculosis H37Rv; 2, M. tuberculosis H37Ra 3, M. tuberculosis Erdman; 4, M. bovis AN5; 5, M. bovis BCG (Japan); 6, M. bovis BCG (Copenhagen); 7, M. bovis IC 378; 8, M. bovis IC 379; 9, M. bovis IC 380; 10, M. bovis IC 381; 11, M. bovis IC 382; 12, PCR molecular weight marker. Panel D, RFLP poly-acrylamide gel analysis of 645 bp amplicon digested with HpaII (lanes 1-3) and HaeIII (lanes 6-9): Lane 1, M. tuberculosis H37Rv; 2, M. tuberculosis H37Ra; 3, M. bovis BCG; 4, Negative control; M, 100 bp Molecular weight marker; 5, M. tuberculosis H37Rv; 6, M. tuberculosis H37Ra; 7, M bovis BCG; 8, M bovis AN5.

FIG. 5 Nucleotide sequence alignment of hupB gene of M. tuberculosis and M. bovis:

The nucleotide sequence of the C-terminal region (326-676 bp) of hupB gene of standard strains of M. tuberculosis and M. bovis and clinical isolates of M. bovis has been aligned using GCG software. A deletion of 27 bp was seen in hupB sequence of all M. bovis strains. The 9 deleted amino acids (KAATKAPAR) between 385 to 411 bp with respect to M. tuberculosis are shown in single letter code on the first line. Numbers in brackets refer to nucleotide position in hupB (Rv2986c). The M. bovis strain numbers are given on the left.

FIG. 6 Nested PCR Profile of M. tuberculosis and M. bovis Standard and Cattle derived isolates:

The nested PCR amplified fragments of the mycobacterial strains were electrophoresed on native 8% polyacrylamide gel, shown in Lanes 1 negative control; 2 molecular markers; 3 M. tuberculosis (H37Rv); 3 Cattle isolate identified as M. tuberculosis; 4 Cattle isolate identified as M. bovis; M. bovis (ICC380); and 5 M. tuberculosis (JALMA, Agra, Isolate).

### DESCRIPTION OF THE INVENTION

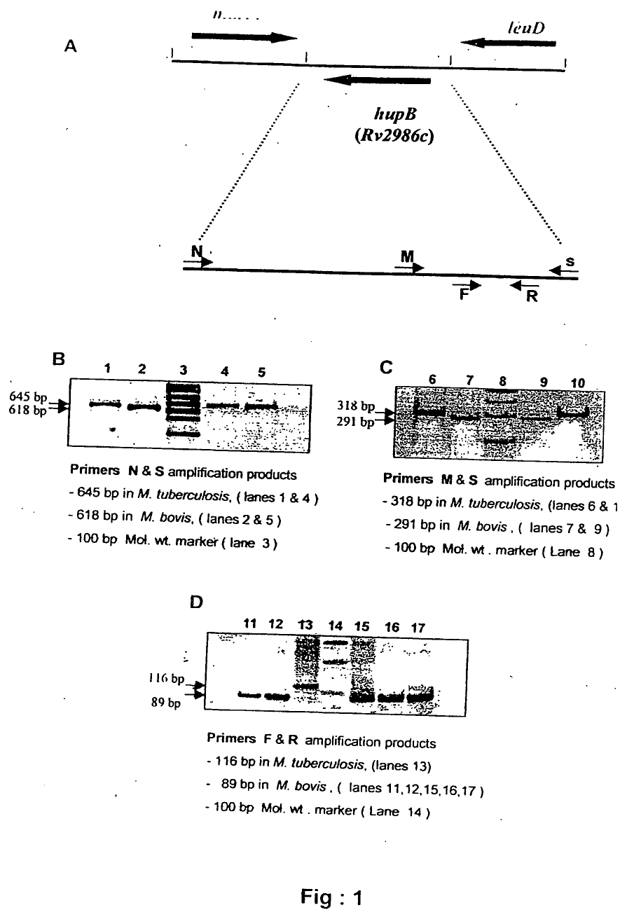

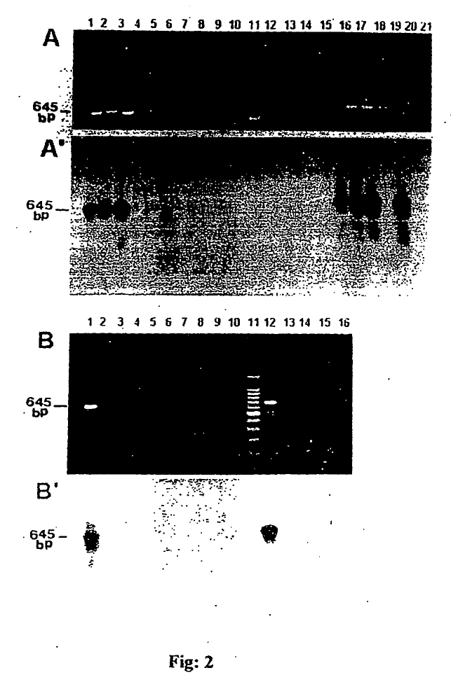

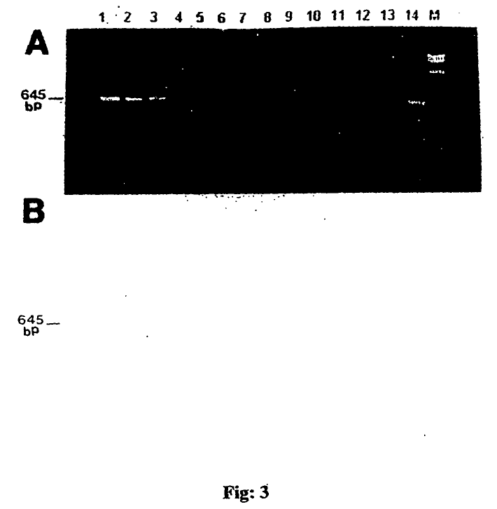

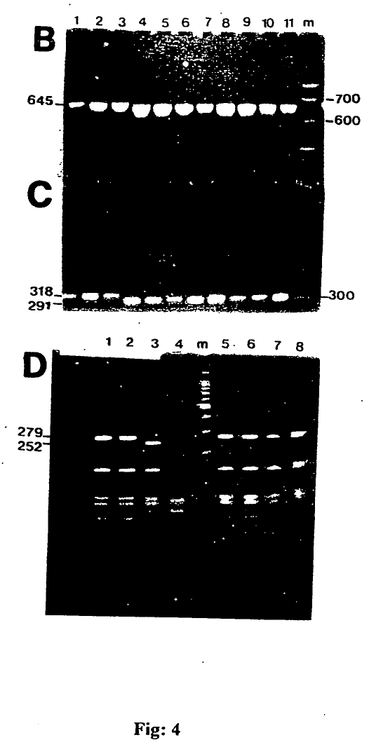

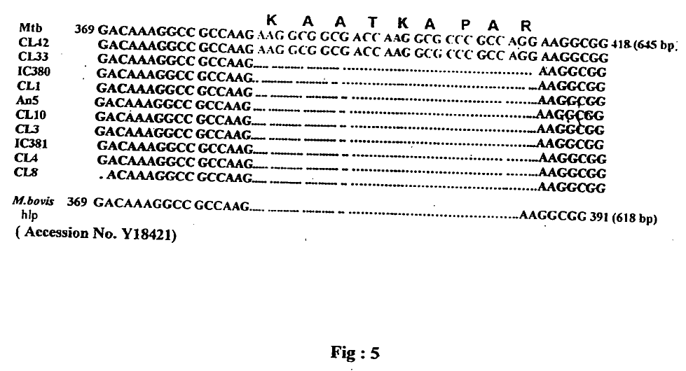

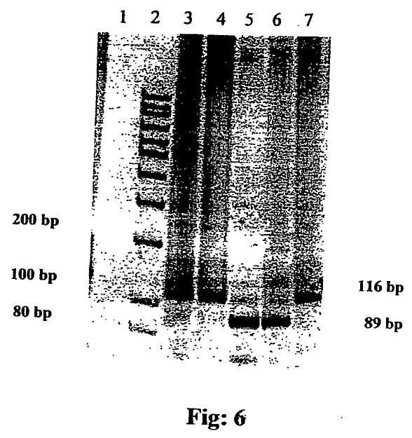

A method for differentiating of the hupB gene of M. tuberculosis and M. bovis. The size variability of the hupB gene was determined using 3 sets of primers (FIG. 1, Table II):

An embodiment the present invention provides for oligonucleotide primers which are specific amplification of the hupB gene of Mycobacterium species selected from the group consisting of Seq ID No. 1, Seq ID No. 2, Seq ID No. 3, Seq ID No. 4, Seq ID No. 5.

Another embodiment is a method for differentiating Mycobacterium species based on target gene encoding for histone like proteins such as hup B.

Yet another embodiment is wherein Mycobacterium tuberculosis, and or Mycobacterium bovis species is selected from a group of genetically related Mycobacteria and from unrelated microorganisms.

Another embodiment is a method wherein the pair of oligonucleotide primers comprising of Seq ID No. 1 and Seq ID No. 2; Seq ID No. 3 and Seq ID No. 2; Seq ID No. 4 and Seq ID No. 5, wherein the amplified fragments are detected by ethidium bromide staining or DNA probe hybridization.

Another embodiment is a differentiating method comprising of designing primers Seq ID No. 1, Seq ID No. 2, Seq ID No. 3, Seq ID No. 4, Seq ID No. 5, to amplify a part of the said hup B gene from Mycobacterium tuberculosis and Mycobacterium bovis. Amplifying a part of the target gene encoding for histone like proteins such as hup B of Mycobacterium species using said DNA as a template in a polymerase chain reaction with a pair of oligonucleotide primers.

Analyzing and validating the size of the amplified fragments. Determining the complete sequence of the said amplified fragments. Inferring from the sequence whether it is M. tuberculosis or M. bovis.

Another embodiment is a method wherein the DNA probe consists of sequence ID No. 6 or sequence ID No. 7 or a complement thereof tagged with a detectable label.

Another embodiment is a method wherein the step of differentiation consists in determining the smaller size of the amplified fragment obtained from Mycobacterium bovis.

Another embodiment is a method wherein the PCR amplified fragment in Mycobacterium bovis was 618 bp.

Another embodiment is a method wherein the PCR amplified fragment in Mycobacterium tuberculosis was 645 bp.

Another embodiment is a method wherein the PCR amplified fragment in Mycobacterium bovis was 291 bp.

Another embodiment is a method wherein the PCR amplified fragment in Mycobacterium tuberculosis was 318 bp.

Another embodiment is a method wherein the PCR amplified fragment in Mycobacterium bovis was 89 bp

Another embodiment is a method wherein the PCR amplified fragment in Mycobacterium tuberculosis was 116 bp.

Another embodiment is a method wherein the PCR amplified fragment in Mycobacterium bovis was 27 bp smaller than that of Mycobacterium tuberculosis.

Another embodiment is a method wherein differentiating M. tuberculosis and M. bovis comprising the steps of amplifying a part of the target hup B gene from M. tuberculosis and M. bovis in a polymerase chain reaction with primers Seq. ID No.1 and Seq. ID No.2. Restricting the amplified fragment with Hpa II restriction enzyme to produce restricted fragments. Separating the restricted fragments by electrophoresis on 12% polyacrylamide gel and detecting the restricted fragments by staining with ethidium bromide.

Another embodiment is a method wherein the restricted fragment in M. tuberculosis was 280 bp and 150 bp.

Another embodiment is a method wherein the restricted fragment in M. bovis was 253 bp and 150 bp.

Another embodiment is Hup B gene (Seq ID No. 8) substantially as herein described a process as in preceding embodiments has been substantially described.

Another embodiment is Hup B gene (Seq ID No. 7) substantially as herein described a process as in preceding embodiments has been substantially described.

The method for differentiating Mycobacterium species amplifying a part of the hup B target gene encoding for histone like proteins of Mycobacterium species using said DNA as a template in a polymerase chain reaction with a pair of oligonucleotide primers. Detecting said amplified fragment of the hup B gene to detect the presence of Mycobacterial species or not and differentiating Mycobacterium tuberculosis from Mycobacterium bovis based on the size of the amplified fragment.

Oligonucleotide primers for specific amplification of the hupB gene of Mycobacterium species selected from the group consisting of Seq ID No. 1, Seq ID No. 2, Seq ID No. 3, Seq ID No. 4, Seq ID No. 5. A method for differentiating Mycobacterium species based on target hup B gene. Using DNA from culture or from clinical samples as a template in a polymerase chain reaction. Detecting the said amplified fragment of the hup B gene to determine whether Mycobacterium species is present and differentiating Mycobacterium tuberculosis from Mycobacterium bovis based on the size of the amplified fragment.
1) hupB gene target DNA Primers N, Seq ID No. 1 (5′ ggagggttgggatgaacaaagcag 3′) and S, Seq ID No. 2 (5′ gtatccgtgtgtcttgacctatttg 3′) were used to amplify hupB gene sequences. The expected size of the amplicon was 645 bp in case of M. tuberculosis, and 618 bp in case of M. bovis respectively.

PCR-RFLP: A method for differentiating M. tuberculosis and M. bovis. Comprising the steps of amplifying the target hup B gene from M. tuberculosis and M. bovis in a polymerase chain reaction with primer pair Seq. ID No.1 and Seq. ID No.2/Seq. ID No.3 and Seq. ID No.2. Restricting the amplified fragment with Hpa II restriction enzyme to produce restricted fragments. Separating the restricted fragments by electrophoresis on 12% polyacrylamide gel
2) The C-terminal portion of the gene: was amplified by using:
   (i) internal primer M, Seq ID No. 3 (5′ gcagccaagaaggtagcgaa 3′) with S, Seq ID No. 2 (5′ gtatccgtgtgtcttgacctatttg 3′), the expected amplicon was ˜318 bp, (FIG. 1). The expected size of the amplicon was 318 bp in case of M. tuberculosis, and 291 bp in case of M. bovis respectively.

Nested PCR: A method for differentiating M. tuberculosis and M. bovis. Comprising the steps of amplifying a part of the target hup B gene from M. tuberculosis and M. bovis in a polymerase chain reaction. The PCR fragment obtained with primers Seq. ID No.1-N and Seq. ID No.2-S was used as target DNA in nested PCR. The C-terminal portion of the gene was also amplified by using Seq.ID. No.4-F and Seq.ID. No.5-R the expected amplicon was ˜116 bp in case of M.tuberculosis and 89 bp in case of M.bovis,
(ii) using primers F, Seq ID No. 4 (5′ ccaagaaggcgacaaagg3′) with R, Seq ID No. 5. (5′ gacagctttcttggcggg3′). The expected size of the amplicon was 116 bp in case of M. tuberculosis, and 89 bp in case of M. bovis respectively.

Sequencing of PCR amplified fragments: Analyzing and validating the size of the amplified fragments of the hup B gene by determining the complete sequence of the amplified fragments. Inferring from the sequence whether it is M. tuberculosis or M. bovis. The step of differentiation consists in determining the smaller size of the amplified fragment obtained from Mycobacterium bovis. The PCR amplified fragment obtained using primers Sequence ID No.1 and 2 in case of Mycobacterium bovis was 618 bp. The PCR amplified fragment in Mycobacterium tuberculosis was 645 bp. Whereas the PCR amplified fragment obtained using primers Sequence ID No.3 and 2 in Mycobacterium bovis was 291 bp and 318 bp in case of Mycobacterium tuberculosis. The PCR amplified fragment obtained using primers Sequence ID No.4 and 5 in Mycobacterium bovis was 89 bp. and 116 bp. in Mycobacterium tuberculosis respectively. The PCR amplified fragment in Mycobacterium bovis was 27 bp smaller than that of Mycobacterium tuberculosis.

Mycobacterial DNA extracted from M. tuberculosis and M. bovis were used. PCR amplified fragment was obtained in both M. tuberculosis and M. bovis. However the amplicon obtained in case of M. bovis was slightly smaller than that obtained in case of M. tuberculosis. This difference was confirmed by analyzing over 50 M. tuberculosis and M. bovis strains collected from diverse sources (Table I). The DNA extracted from 3 standard strains and 4 clinical isolates of M. tuberculosis and M. bovis (BCG) were included for amplification using the hupB primers (N, Seq ID No. 1 and S, Seq ID No. 2,/M, Seq ID No. 3 and S, Seq ID No. 2, Table II). The difference in the size of amplicons obtained in case of M. tuberculosis and M. bovis was validated by RFLP (FIG. 4D) and confirmed by sequencing of the PCR fragments, (FIG. 5). The PCR fragments of the two mycobacteria were digested with HaeIII and HpaII and analyzed on 12% non-denaturing gel. Digestion of the 645 bp fragment with HpaII revealed that a ˜250 bp fragment was seen in case of M. bovis compared to the band of ˜280 bp size obtained in case of M. tuberculosis, (FIG. 4D). Analyzing the sequence of the PCR fragments showed that in M. bovis there was a deletion of 27 bp corresponding to 9 amino acids, (FIG. 5). As a result of this deletion the PCR amplicon obtained in case of M. bovis was 618 bp, 27 bp smaller than the PCR fragment obtained in case of M. tuberculosis (645 bp), (FIGS. 4B,C).

Results, obtained with the amplicon generated in the C-terminal portion of the gene using M and S primers on digestion with HpaII, showed differences matching to the differences seen in case of the PCR fragment obtained using the hupB primers (Seq ID No. 1-N and Seq ID No. 2-S) indicating that the PCR-RFLP assay utilizing either the PCR fragment obtained using the hupB primers (N, Seq ID No. 1-N and S, Seq ID No. 2-S)/the C terminal primers (Seq ID No. 3-M and S, Seq ID No. 2-S) did distinguish between M. tuberculosis and M. bovis.

The utility of the hupB gene as a target in diagnosis and identification pathogenic mycobacteria in bovine tuberculosis has been demonstrated, (Table IV-VII). The sensitivity and specificity of the assay showed remarkable improvement with the adoption of the nested PCR technique in clinical samples, targeting the C-terminal part of the hupB gene, (FIG. 6) and (Table VI-VIII).

### EXAMPLES

Bacterial strains: The mycobacterial strains as well as non-mycobacterial strains used in the study have been listed in Table I. In all 80 mycobacterial strains were included in the study besides 10 non-mycobacterial species. Of the 80 mycobacterial isolates included 55 were members of the MTB complex, (M. tuberculosis—25, M. bovis—25, M. microti—3, and 1 each of M. africanum and M. canetti. The details of the M. bovis strains included are as follows: 7 from infected cattle housed in the Central Military Veterinary Laboratory, Meerut, India, 9 from National Mycobacterial Repository, JALMA, Agra India, 2 each from Netherlands and Argentina and 3 human isolates from the Netherlands (Drs. J. D. A. van Embden and D. van Soolingen).

Processing of Bacilli for Specificity Analysis

All the mycobacterial and non-mycobacterial strains grown on solid media (LJ slants all mycobacterial species), LB agar (E. coli) nutrient agar (Aspergilus niger, Nocardia asteriodes, Pseudomonas aeruoginosa, Klebsiella pneumoniae) or blood agar (Corynebacterium diphtheriae, Streptococcus pneumoniae) were scraped with the help of sterile toothpicks and re-suspended in sterile distilled water containing 0.1% Triton X-100. Re-suspended bacilli were boiled at 100° C. for 20 minutes and an aliquot (2 μl) was used for PCR.

PCR Analysis:

1) 23S rDNA target: Primers: C, Seq ID No.9(5′ gtgagcgacgggatttgcctat 3′) and L, Seq ID No. 10(5′ accacccaaaaccggatcgat 3′) were used to detect the presence of DNA from organisms belonging to genus Mycobacterium. The expected size of the amplicon was 174 bp (Verma et. al., 1994; Dasgupta et al., 1998).
2) hupB DNA target: Primers N, Seq ID No. 1 (5′ ggagggttgggatgaacaaagcag 3′) and S, Seq ID No. 2 (5′ gtatccgtgtgtcttgacctatttg 3′) were used to amplify hupB gene sequences. The expected size of the amplicon was ˜645 bp (Table II, FIG. 1) in M. tuberculosis and 618 bp in M. bovis.

Each reaction (20 μl) contained 1.5 mM MgCl₂, 0.5 μM of primers, 200 μM. dNTPs, 10 mM Tris-HCl (pH 8.8 at 25° C.), 50 mM KCl, 0.08% Nonidet P40, and 0.5 Units of Taq DNA Polymerase. The PCR reaction was subjected to initial denaturation at 94° C. for 10 min., and 35 cycles of each of 1 min at 94° C., 1 min., at 63° C. and 1 min at 72° C. followed by final extension at 72° C. for 30 mins. The fragments were analyzed on a 1.2% agarose gel and stained with ethidium bromide.

The C-terminal portion of the gene was amplified by using M, Seq ID No. 3 (5′ gcagccaagaaggtagcgaa 3′) with S, Seq ID No. 2 (5′ gtatccgtgtgtcttgacctatttg 3′), the expected amplicon was ˜318 bp.

Nested PCR: DNA extracted from clinical samples/cultivated mycobacteria were processed for PCR with primers Seq.ID. No.1-N and Seq.ID. No.2-S. The PCR product obtained using the primers Seq.ID. No.1-N and Seq.ID. No.2-S was used as target DNA in nested PCR.

Each reaction (40 μl) contained 2.5 mM MgCl₂, 0.5 μM of primers, 200 μM dNTPs, 10 mM Tris-HCl (pH 8.8 at 25° C.), 50 mM KCl, 0.08% Nonidet P40, and 0.5 Units of Taq DNA Polymerase. The PCR reaction was subjected to initial denaturation at 95° C. for 10 min., and 35 cycles of 1 min at 94° C., 1 min., and 30 seconds at 59° C. and final extension at 72° C. for 7 mins. The fragments were analyzed on a 3.5% agarose gel/8% non-reducing polyacrylamide gel and stained with ethidium bromide. The C-terminal portion of the gene was also amplified by using Seq.ID. No.4-F (5′ ccaagaaggcgacaaagg3′) with Seq.ID. No.5-R (5′ gacagctttcttggcggg3′), the expected amplicon was ˜116 bp in case of M.tuberculosis and 89 bp in case of M.bovis, (Table II, FIG. 1).

Southern Hybridization: The PCR amplicons resolved on the agarose gel were transferred on to nitro-cellulose membrane (Southern, 1975). The blots were then hybridized with α-³²P labeled 645 bp hupB (Seq ID No.6) gene probe from M. tuberculosis, (PstI & NcoI digest from the plasmid pHLPMT/probe generated by PCR using N (Seq ID No.1-N) and S (Seq ID No.2-S) primers and M. tuberculosis, DNA.).

Restriction Fragment Length Polymorphism:

hupB amplified sequences were digested with HpaII restriction enzyme and the fragments were analyzed on a 12% non-denaturing polyacrylamide gel. The gel was strained with ethidium bromide and DNA fragments were visualized under ultraviolet light.

DNA Sequencing Analysis: The PCR fragments were sequenced by the Sanger's dideoxy chain termination method (Sanger et al., 1977) using Sequenase Ver 2.0 sequencing kit, α³⁵ SdATP and forward/reverse universal M13 primers or internal primers of hupB, according to the manufacturer's instructions. The DNA template was alkali denatured and annealed to the primers at −70° C. for 1 hour. The GC rich mycobacterial DNA was mixed with 0.5 μg of single strand binding protein prior to labeling. The protein was digested with proteinaseK 0.1 μg at 68° C. for 20 mins., following termination of the labeling reaction. The reactions were electrophoresed on a 6% urea-polyacrylamide gel in 1×TBE at 70 W for a suitable time period. The gel was fixed with acetic acid (10%) and methanol (30%) dried and autoradiographed. The PCR fragments obtained in standard strains and isolates were also sequenced commercially by Microsynth, Switzerland.

The specificity of the PCR assay; DNA from 16 mycobacterial and 10 non-mycobacterial species were used as target to establish the specificity of the PCR assay, (Table 1). The DNA extracted from 3 standard strains and 4 clinical isolates of M. tuberculosis and M. bovis (BCG) were included for amplification using the hupB primers (Seq ID No. 1-N, and Seq ID No. 2-S, Table II, FIG. 1). Only in case of M. tuberculosis H37Rv, H37Ra, M. bovis BCG and 5 clinical isolates of M. tuberculosis (lanes:1, 2,3,16,17,18 and 20, in FIG. 2A and lanes 1 and 12 in FIG. 2B) the expected 645 bp fragment was obtained in case of M. tuberculosis and 618 bp in case of M. bovis. No amplification was seen with M. microti, M. africanum of the MTB complex, M. leprae, MAIS complex and other mycobacterial species (rapid and slow growers) including Corynebacterium diphtheriae and Nocardia asteriodes that together make the Corynebacteria, Nocardia and Mycobacteria (CNM) group. Amplification was also not seen in other non-mycobacterial species (FIG. 2B). The authenticity of the amplified fragment was confirmed by hybridization with α-³²P labeled 645 bp fragment (Seq ID No.6) (FIGS. 2A′ and B′). This confirmed that no other amplification was obtained with any other template DNA that could have been missed by ethidium bromide staining alone. Thus the 5′ and 3′ primers of hupB are specific for M. tuberculosis and M. bovis.

Sensitivity of hupB gone based PCR assay: The sensitivity of DNA PCR amplification (level of detection) was established by adding serial dilutions of mycobacterial DNA (1 ng to 1 fg) in the PCR reaction using primers Seq ID No. 1-N and Seq ID No. 2-S. It was seen that by ethidium bromide staining alone the detection limit was 50 pg and by hybridization the detection limit increased to 500 fg (FIGS. 3A and B). This was equivalent to the detection of 5000 and 50 genome equivalents respectively.

RFLP of PCR Amplicons of hupB gene derived from M. tuberculosis and M. bovis: DNA from different isolates of M. tuberculosis and M. bovis (listed in Table I) were amplified using Seq ID No. 1-N and Seq ID No. 2-S primers (645 bp fragment, Table II) and (ii) Seq ID No. 3-M (internal primer) and Seq ID No. 2-S (318 bp fragment, FIG. 4C, Table II, FIG. 1). PCR amplicons obtained from the DNA of M. bovis strains (lanes 4-11, FIGS. 4B and 4C) were smaller in size as compared to the PCR amplicons obtained from the M. tuberculosis strains (lanes 1-3, FIGS. 4B and 4C). The results of the PCR assay with the 2 sets of primers have been summarized in Table III.

In order to confirm the difference in 645 and 618 bp PCR fragment sizes, the amplicons were digested with HpaII and HaeIII (FIG. 4D). The digested fragments were analyzed on 12% non-denaturation polyacrylamide gel. Digestion of 645 bp fragment with HpaII clearly revealed that in case of M. bovis a ˜250 bp (FIG. 4D, lane 3) fragment obtained was smaller in size compared to the ˜280 bp bands obtained with M. tuberculosis H37Ra & H37Rv (FIG. 4D, lanes 1 and 2). No differences were perceived with HaeIII digestion, (FIG. 4D, lanes 5-8). Results, obtained with the amplicon (318 bp) generated in the C-terminal portion of the gene using Seq ID No. 3-M and Seq ID No. 2-S primers on digestion with HpaII, showed similar differences (results not shown) indicating that the PCR-RFLP assay did distinguish between M. tuberculosis and M. bovis strains.

Sequencing of PCR Amplified Fragment: PCR amplicons obtained from DNA of standard strains of M. bovis and M. tuberculosis including local isolates of M. bovis derived from cattle were sequenced. The PCR amplicons 618 and 645 bp (obtained using Seq ID No. 1-N and 2-S), 318 and 291 bp (obtained using Seq ID No. 3-M and 2-S), 116 and 89 bp (obtained using Seq ID No. 4-F and 5-R) were sequenced to confirm the size differences. Sequence analysis indicated that in M. bovis there was a deletion of 27 bp (9 amino acids) in frame after 128^{th} codon in the C terminal part of the gene (FIG. 5,). The histone like gene sequence of M. bovis (Accession No. Y18421) and M. tuberculosis (Accession No. P95109) has been submitted to the NCBI data base.

Advantages of PCR, RFLP and Nested PCR Assay:

1) Unlike currently available assays the assay provides a method for direct detection and identification of human pathogenic mycobacteria in clinical samples, dairy and meat products. The assay enables the identification of human pathogenic mycobacteria belonging to the Mycobacterium tuberculosis Complex.
2) The method described herein has the unique advantage over existing methods as a means for not only simultaneous detection but also precise identification of two intimately related mycobacteria namely M. tuberculosis and M. bovis.
3) The PCR, RFLP and nested PCR technique described herein takes advantage of the unique novelty of the Hup B gene as a target. The novel primer pairs (Seq. ID. No. 1 & 2; 3 & 2; 4 & 5) designed facilitates the specific amplification of the Hup B gene in two known pathogenic mycobacteria namely M. tuberculosis and M. bovis,
4) The size and sequence difference of the amplified fragments permits their reliable identification, which hitherto was not possible by all other reported methods.

5) More importantly the method described enables the investigator to detect and diagnose dual infection in clinical samples caused by pathogenic mycobacteria other than M. tuberculosis such as M. bovis.

| | | |
|---|---|---|
| M. tuberculosis | H37Rv, H37Ra, Erdman, P8473, P8497, | a, b, c, d,, g n |
| (Human isolates) | C1207, C1084, 779634, ICC107, ICC120, | |
| | ICC22, ICC238, ICC136, ICC37, ICC247, | |
| | ICC16, ICC235, ICC145, ICC06, ICC11, | |
| | ICC85, ICC95, CSU-17, CSU-27, CSU-20 | |
| M. bovis (Cattle Isolates) | T11, AN5, IC378, IC379, IC380, IC381, | d, o, p, q |
| | IC382, ICC388, ICC391, 117, 126, 73, 130, | |
| | CL1, CL3, CL4, CL8, CL10, CL33, | |
| | CL42, Japanese* & Copenhagen* | |
| Human Isolates | 6, 47, 85, | o |
| M. canetti | 116 | o |
| M. africanum | 81543 | e, g |
| M. microti | OV254, T14, N5 | d, f |
| M. gastri | TMC1456 | b |
| M. chelonae | TMC191, J31 | b |
| M. vaccae | IND123 | b |
| M. avium | NCTC8562, ICC192 | d |
| M intracellulare | TMC1302, N25, N8 | d |
| M. scrofulaceum | TMC1302, MAC29 | d |
| M. gordonae | TMC1324 | d |
| M. fortuitum | 5J32, ICC420, ICC419, ICC417, ICC416 | g, d, l |
| M. smegmatis | ATCC27204, LR222, N18 | b, d |
| M. phlei | ND124, N14 | b |
| M. Kansasii | 1201 | c |
| M. leprae | Tissue Biopsy | d |
| M. simae | IN7 | d |
| Corynebacterium diphtheriae | Clinical isolate | h |
| Streptococcus β-haemolyticus | Clinical isolate | h |
| Staphylococcus aureus | Clinical isolate | h |
| Pseudomonas aeruginosa | Clinical isolate | h |
| Klebsiella pneumoniae | Clinical isolate | h |
| Nocardia asteriodes | MTCC274 | i |
| Aspergillus fumigatus | Soil isolate | j |
| Aspergillus niger | Soil isolate | j |
| Candida albicans | Clinical isolate | k |
| Escherichia coli | DH5α, BL21 (DE3) | m |

a - P. S. Murthy, UCMS, University of Delhi, India;
b - N. K. Jain, NDTC, New Delhi, India;
c - C. N. Paramasivan, TRC, Chennai, India;
d - V. M. Katooh, JALMA, Agra, India;
e - Y. M. Yates, Public Health Laboratory, Dulwich Hospital, London, UK;
f - P. Draper, NMR, Mill Hill, London, UK;
g - Kathleen Eisenach, University of Arkanasa, USA;
h - Dept. of Microbiology, AIIMS, New Delhi, India;
i - Microbiological Type Culture Collection, IMTECH, Chandigarh, India;
j - Shivkumar, Anna University, Chennai, India;
k - Z. U. Khan, V.P. Chest Institute, Delhi, India;
l - Jack Crawfort, CDC, Atlanta, GA, USA;
m - GIBCO BRL, USA;
n - Suman Laal, VA Medical Center, NY U, School of Medicine, New York, USA;
o - J. D. A. van Embden, Netherlands;
p - Central Military Veterinary Laboratory, Meerut, India;
q - Dept. of Paediatrics, AIIMS, New Delhi, India;
*Human vaccine strain;
Numbers in bold - human isolates.

TABLE II
Primers Used for Amplification of hupB Mycobacterial DNA Target

| | | Target | | PCR |
|---|---|---|---|---|
| Primer | | hup B | | Product |
| Pair | Sequence of Primer | gene | Mycobacteria | Size |
| Seq. Id. No. 1-N | (5′ggagggttgggatgaacaaagcag 3′) | Whole | M. tuberculosis | 645 bp |
| Seq. Id. No. 2-S | (5′ gtatccgtgtgtcttgacctatttg 3′) | gene | M. bovis | 618 bp |
| Seq. Id. No. 3-M | (5′ gcagccaagaaggtagcgaa 3′) | C terminal | M. tuberculosis | 318 bp |
| Seq. Id. No. 2-S | (5′ gtatccgtgtgtcttgacctatttg 3′), | | M. bovis | 291 bp |
| Seq. Id. No. 4-F | (5′ ccaagaaggcgacaaagg3′) | C terminal | M. tuberculosis | 116 bp |
| Seq. Id. No. 5-R | (5′ gacagctttcttggcggg3′). | | M. bovis | 89 bp |

Sequence
<213> OrganismName: Mycobacteria
<400> PreSequenceString:

| | |
|---|---|
| VKPTSVPAFR PGAQFK | 16 |

```
<212> Type: PRT
<211> Length: 16
      SequenceName: seq id no. 6 (Change to 8)
      SequenceDescription:
Sequence
<213> OrganismName: hup B - M. tuberculosis, Rv2986c, Accession No. P95109
<400> PreSequenceString:
```

| | |
|---|---|
| atgaacaaag cagagctcat tgacgtgctc acacagaaat tgggctcgga ccgtcggcag | 60 |
| gcgaccgccg ccgtcgagaa tgtcgttgac acgattgtgc gtgcggtaca caaaggcgac | 120 |
| agcgtcacca ttaccgggtt cggtgtgttc gaacagcgtc gccgcgcggc tcgagtggcc | 180 |
| cgcaatccgc gtaccggcga gacagtaaag gtgaagccga cgtcggtgcc ggcgttccgc | 240 |
| ccgggcgcgc aattcaaagc ggttgtgtct ggcgcgcagc gtctcccggc agaaggaccc | 300 |
| gctgttaagc gtggtgtggg ggccagtgca gccaagaagg tagcgaagaa ggcacctgcc | 360 |
| aagaaggcga caaaggccgc caagaaggcg gcgaccaagg cgcccgccag gaaggcggcg | 420 |
| accaaggcgc ccgccaagaa agcggcgacc aaggcgcccg ccaagaaagc tgtcaaggcc | 480 |
| acgaagtcac ccgccaagaa ggtgaccaag gcggtgaaga agaccgcggt caaggcatcg | 540 |
| gtgcgtaagg cggcgaccaa ggcgccggca aagaaggcag cggccaagcg gccggctacc | 600 |
| aaggctcccg ccaagaaggc aaccgctcgg cggggtcgca aatag | 645 |

```
<212> Type: DNA
<211> Length: 645
      SequenceName: Seq id no. 7 (Change to 6)
      SequenceDescription:
Sequence
<213> OrganismName: Hlp of Mycobacterium bovis, Accession No. Y18421
<400> PreSequenceString:
```

| | |
|---|---|
| atgaacaaag cagagctcat tgacgtgctc acacagaaat tgggctcgga ccgtcggcag | 60 |
| gcgaccgccg ccgtcgagaa tgtcgttgac acgattgtgc gtgcggtaca caaaggcgac | 120 |
| agcgtcacca ttaccgggtt cggtgtgttc gaacagcgtc gccgcgcggc tcgagtggcc | 180 |
| cgcaatccgc gtaccggcga gacagtaaag gtgaagccga cgtcggtgcc ggcgttccgc | 240 |
| ccgggcgcgc aattcaaagc ggttgtgtct ggcgcgcagc gtctcccggc agaaggaccc | 300 |
| gctgttaagc gtggtgtggg ggccagtgca gccaagaagg tagcgaagaa ggcacctgcc | 360 |
| aagaaggcga caaaggccgc caagaaggcg gcgaccaagg cgcccgccaa gaaagcggcg | 420 |
| accaaggcgc ccgccaagaa agctgtcaag gccacgaagt cacccgccaa gaaggtgacc | 480 |
| aaggcggtga agaagaccgc ggtcaaggca tcggtgcgta aggcggcgac caaggcgccg | 540 |
| gcaaagaagg cagcggccaa gcggccggct accaaggctc ccgccaagaa ggcaaccgct | 600 |
| cggcggggtc gcaaatag | 618 |

```
<212> Type: DNA
<211> Length: 618
      SequenceName: Seq id no. 8 (Change to 7)
      SequenceDescription:
Sequence
<213> OrganismName: 23S- Genus Mycoabcteria-C
<400> PreSequenceString:
```

| | |
|---|---|
| gtgagcgacg ggatttgcct at | 22 |

```
<212> Type: DNA
<211> Length: 22
      SequenceName: seq id no. 9
      SequenceDescription:
Sequence
<213> OrganismName: 23S-Genus Mycobacteria-L
<400> PreSequenceString:
```

| | |
|---|---|
| accacccaaa accggatcga t | 21 |

```
<212> Type: DNA
<211> Length: 21
      SequenceName: Seq id no. 10
      SequenceDescription:
```

TABLE III A
Representative results of hupB PCR Assay
with Strains of M. tuberculosis
M. tuberculosis

| | | PCR Amplified Product |
|---|---|---|
| | | Obtained |
| Strain | Source | 645/318 bp |
| H37Rv | ATCC^{a} | +/+ |
| H37Ra | ATCC | +/+ |
| Erdman | ATCC | +/+ |
| 779634 | Human isolate^{b} | +/+ |
| P8473 | Human isolate | +/+ |
| P8497 | Human isolate | +/+ |
| C1207 | Human isolate | +/+ |
| C1084 | Human isolate | +/+ |

^{a}Dr. Kathleen Eisenach, University of Arkansas, USA
^{b}Dr. C. N. Paramasivan, Tuberculosis Research Centre, Chennai, India

TABLE III B
Representative results of hupB PCR Assay with Strains of M. bovis
M. bovis

| | | PCR Amplified |
|---|---|---|
| | | Product |
| | | Obtained |
| Strain | Source | 618/291 bp |
| AN5 | Cattle isolate^{c} | +/+ |
| IC378 | Cattle isolate | +/+ |
| IC379 | Cattle isolate | +/+ |
| IC380 | Cattle isolate | +/+ |
| IC381 | Cattle isolate | +/+ |
| IC382 | Cattle isolate | +/+ |
| 117 | Cattle isolate (Argentina)^{d} | +/+ |
| 126 | Cattle isolate (Argentina) | +/+ |
| BCG | Japanese^{e} | +/+ |
| BCG | Copenhagen | +/+ |

^{c}Dr. V. M. Katoch, JALMA, Agra, India;
^{d}Dr. J. D. A. van Embden, Netherlands
^{e}Pediatrics Dept. of AIIMS, New Delhi.

TABLE IV
Results of the Direct PCR assay carried out with
Bovine Samples

| | Detection of M tb Complex | | |
|---|---|---|---|
| | by the PCR Assay | | |
| Samples | Number Tested | Number Positive | Percent |
| Lymph Gland Biopsy | 89 | 21 | 23.6 |
| Blood (Heparinsed) | 89 | 01 | 01.1 |
| Pharyngeal Swab | 89 | 02 | 02.2 |
| Faeces | 89 | 02 | 02.2 |
| Rectal Pinch | 89 | 03 | 03.4 |
| Milk | 89 | 11 | 12.4 |
| Total Tested | 534 | 40 | 07.5 |

The following bovine samples were found to be appropriate for the PCR based assay for detection of bovine tuberculosis: Lymph Gland Biopsy and Milk were found to be the best (Chi square test, p value < 0.05 at significance level, (SAS 8.0, Statistical Software).

TABLE V
Comparative Analysis Of Clinical & AFB Status
of Cattle With Direct PCR Results

| | |
|---|---|
| Clinical Status | Number Positive For |

| Category | Number | Acid Fast Bacilli | PCR |
|---|---|---|---|
| A | 17 | 13 | 07 |
| B | 12 | Nil | Nil |
| C | 20 | 12 | 09 |
| D | 20 | 05 | 08 |
| E | 20 | Nil | 02 |
| Total | 89 | 30 | 26 |
| | | (33.7%) | (29.2%) |

A - Tuberculin Positive with Clinical Signs of Tuberculosis
B - Tuberculin Positive, Apparently healthy Animal
C - Tuberculin Negative with Clinical Signs of Tuberculosis
D - Tuberculin Negative Apparently healthy Animal
E - Animal Infected with non-mycobacterial infection
Among the clinical categories of animals investigated, bovine tuberculosis was detected least in animals infected with non-mycobacterial micro-organisms (Category E), compared to all other categories (p < 0.05, (Chi square test, p value < 0.05 at significance level, SAS 8.0, Statistical Software).

TABLE VI
Nested PCR based Identification of Pathogenic
Mycobacteria in Cattle Derived Samples.

N-PCR based
Identification of^{b}

| Samples^{a} | M. tuberculosis | M. bovis |
|---|---|---|
| Lymph Gland | 15 | 18 |
| Biopsy | | |
| Blood^{c} | 14 | 14 |
| Milk | 26 | 26 |
| Total Tested | 55 | 58 |
| 192 | (28.6%) | (30.2%) |

^{a}64 Samples tested in each category
^{b}Nested PCR for the C terminal region of the hup B gene
^{c}Citrated Blood

TABLE VII
Comparative Analysis Of Clinical & AFB Status
of Cattle With Nested PCR Results

| | |
|---|---|
| Clinical Status | Number Positive For |

| Category | Number | Acid Fast Bacilli | N-PCR |
|---|---|---|---|
| A | 20 | 09 | 19 |
| B | 17 | 03 | 16 |
| C | 09 | 03 | 08 |
| D | 10 | Nil | 07 |
| E | 08 | Nil | 07 |
| Total | 64 | 15 | 57 |
| | | (23.4%) | (89.0%) |

A - Tuberculin Positive with Clinical Signs of Tuberculosis
B - Tuberculin Positive, Apparently healthy Animal
C - Tuberculin Negative with Clinical Signs of Tuberculosis
D - Tuberculin Negative Apparently healthy Animal
E - Animal Infected with non-mycobacterial infection

TABLE VIII
Comparison of Bacteriological and PCR-RFLP/Nested - PCR
based identification of mycobacterial isolates derived from cattle
Identification of Cattle Derived Mycobacterial Isolates

| | |
|---|---|
| Classical | PCR Based Identification |

| Isolate | Criteria | M. tuberculosis | M. bovis |
|---|---|---|---|
| 173 | M. bovis | + | − |
| 315 | M. tuberculosis | + | − |
| 262 | M. bovis | − | + |
| 95 | M. bovis | − | + |
| 101 | M. bovis | + | − |
| 113 | M. bovis | − | + |
| 155 | M. bovis | − | + |
| 28 | M. bovis | − | + |
| 36 | M. bovis | + | − |
| 33 | M. bovis | − | + |

1-24. (canceled)

25. Oligonucleotide primers for specific amplification of a hupB gene of Mycobacterium species selected from the group consisting of Seq ID No. 1, Seq ID No. 2, Seq ID No. 3, Seq ID No. 4 and Seq ID No. 5.

26. A method for differentiating Mycobacterium species based on target gene encoding for histone like proteins such as hupB comprising:
a) obtaining DNA from culture or from clinical samples;
b) amplifying a part of the target gene encoding for histone like proteins such as hupB of Mycobacterium species using said DNA as a template in a polymerase chain reaction with a pair of oligonucleotide primer according to claim 25; and
c) detecting said amplified fragment of the hupB gene to detect the presence of Mycobacterial species or not and differentiating Mycobacterium tuberculosis from Mycobacterium bovis based on the size of the amplified fragment.

27. The method according to claim 26, wherein said Mycobacterium tuberculosis, and or Mycobacterium bovis species is selected from a group of genetically related Mycobacteria and from unrelated microorganisms.

28. The method according to claim 26, wherein the pair of oligonucleotide primers comprises Seq ID No. 1 and Seq ID No. 2.

29. The method according to claim 26, wherein the pair of oligonucleotide primers comprises Seq ID No. 3 and Seq ID No. 2.

30. The method according to claim 26, wherein the pair of oligonucleotide primers comprises Seq ID No. 4 and Seq ID No. 5.

31. The method according to claim 26, wherein the amplified fragments are detected by ethidium bromide staining or DNA probe hybridization.

32. The method as claimed in claim 26, wherein the step of differentiating comprises the steps of:
a) designing a set of primers selected from the group consisting of Seq ID No. 1, Seq ID No. 2, Seq ID No. 3, Seq ID No. 4 and Seq ID No. 5 to amplify a part of said hupB gene from Mycobacterium tuberculosis and Mycobacterium bovis;
b) obtaining DNA from culture or from clinical samples;
c) amplifying a part of the target gene encoding for histone like proteins such as hupB of Mycobacterium species using said DNA as a template in a polymerase chain reaction with a pair of said oligonucleotide primers;
d) analyzing and validating the size of the amplified fragment;
e) determining the complete sequence of said amplified fragments; and
f) inferring from the sequence whether it is Mycobacterium tuberculosis or Mycobacterium bovis.

33. The method according to claim 31, wherein the DNA probe consists of Seq ID No. 7 or Seq ID No. 8 or a complement thereof tagged with a detectable label.

34. The method as claimed in claim 26, wherein the step of differentiation consists in determining the smaller size of the amplified fragment obtained from Mycobacterium bovis.

35. The method according to claim 28, wherein the PCR amplified fragment in Mycobacterium bovis is 618 bp.

36. The method according to claim 28, wherein the PCR amplified fragment in Mycobacterium tuberculosis is 645 bp.

37. The method according to claim 29, wherein the PCR amplified fragment in Mycobacterium bovis is 291 bp.

38. The method according to claim 30, wherein the PCR amplified fragment in Mycobacterium tuberculosis is 318 bp.

39. The method according to claim 29, wherein the PCR amplified fragment in Mycobacterium bovis is 89 bp.

40. The method according to claim 29, wherein the PCR amplified fragment in Mycobacterium tuberculosis is 116 bp.

41. The method according to claim 26, wherein the PCR amplified fragment in Mycobacterium bovis is 27 bp smaller than that of Mycobacterium tuberculosis.

42. The method as claimed in claim 26, wherein differentiating Mycobacterium tuberculosis and Mycobacterium bovis comprises the steps of:
a) amplifying a part of the target hupB gene from Mycobacterium tuberculosis and Mycobacterium bovis in a polymerase chain reaction with primers Seq ID No. 1 and Seq ID No. 2;
b) restricting the amplified fragment with Hpa II restriction enzyme to produce restricted fragments;
c) separating the restricted fragments by electrophoresis on 12% polyacrylamide gel; and
d) detecting the restricted fragments by staining with ethidium bromide.

43. The method according to claim 42, wherein the restricted fragment in Mycobacterium tuberculosis is 280 bp and 150 bp.

44. The method according to claim 42, wherein the restricted fragment in Mycobacterium bovis is 253 bp and 150 bp.

45. A hupB gene comprised of Seq ID No. 8 as claimed in claim 25.

46. A hupB gene comprised of Seq ID. No. 7 as claimed in claim 25.
